(19) United States
(12) Patent Application Publication
(10) Pub. No.: US 2026/0258332 A1
(43) Pub. Date: Sep. 3, 2026

(54) PRINTING OF CELL AGGREGATES

(71) Applicants: Katholieke Universiteit Leuven, Leuven (BE); Poietis, Pessac (FR)

(72) Inventors: Frank Luyten, Kraainem (BE); Ioannis PAPANTONIOU, Leuven (BE); Antonio Iazzolino, Pessac (FR); Bertrand Viellerobe, Merignac (FR); Fabien Guillemot, Preignac (FR)

(21) Appl. No.: 18/877,571

(22) PCT Filed: Jun. 26, 2023
(86) PCT No.: PCT/EP2023/067340

§ 371 Date: Dec. 20, 2024

(30) Foreign Application Priority Data
Jun. 24, 2022 (EP) 22181067.4

(51) Int. Cl. C12M 3/00; C12M 1/26

(52) U.S. Cl. CPC C12M 21/08; C12M 33/00

(57) ABSTRACT

The invention relates to methods of manufacturing a three dimensional osteochondral tissue by Laser Induced Forward Transfer (LIFT) laser printing, comprising the steps of providing a donor bio-ink comprising aggregates of cartilage and/or bone forming cells, transferring cell aggregates to a receiver substrate into a patterned layer of cell aggregates by pulsed laser energy focused on the bio ink, and repeating steps a) and b) to obtain further patterned layers of aggregates on previously deposited layers.

### FIELD OF THE INVENTION

The invention relates to bioprinting of cell aggregates such as cell spheroids to generate three dimensional tissues such as cartilage.

### BACKGROUND OF THE INVENTION

Modular tissue engineering using microtissue spheroids as “cellular building blocks” (CBBs) shows great promise for bioengineering of functional tissue implants. The three-dimensional microenvironment in spheroids allows for cell-cell and after differentiation cell-matrix interaction generating CBBs (microtissues, organoids) that resemble the native tissue with regards to cellular microenvironment and function. Microtissues and organoids have been developed for a variety of tissues e.g. cardiac, cerebral, liver and fracture callus with demonstrated tissue-specific structure and/or function. When assembled, CBBs fuse via cell-cell and cell-matrix interaction resulting in one larger tissue which have been shown to regenerate damaged organs in small animal models, giving potential also for future clinical translation. However, assembly has to a large extent been performed by “bulk” collection of multiple CBBs within a container while an automated and more precise assembly strategy is desired for biofabrication of tissue engineered tissues.

Biomanufacturing technologies have previously been presented for the handling of CBBs but do also entail drawbacks. Extrusion-based bioprinting of spheroids in combination with hydrogels as ink enabled the creation of shapes with micrometer scale, however results to date indicate that structures with low cellular densities are obtained and only limited control over spheroid deposition is achieved. In addition, nozzle clogging when bioprinting spheroids have been reported as a problem. Moreover, the extrusion-based bioprinters focused on “bulk” printing of spheroids while lacking positional accuracy of single spheroid deposition. This aspect was resolved using aspiration for pick-up and deposition of single spheroids. Automated aspiration technique using robotic setups has also been described for large tissue modules. Although the aspiration-based technique allows for single spheroid deposition, nozzle-based techniques are linked with clogging as well as shear stress leading to incapacity to print or spheroid and cell damage while they require large amount of time for transfer of tissues. The nozzle-free printing technique Laser Assisted Bioprinting (LAB) is therefore of interest for CBB printing to circumvent above mentioned drawbacks [Braudy (1969) Laser Writing Proc. IEEE 57 1771-1772; Guillemot et al. (2010) Acta Biomater, 6 2494-2500; Serra & Piqué (2019) Adv. Mater. Technol. 4, 1-33].

Bioprinting is a combination of 3D printing principles and biological morphogenesis constraints. Herein, computer assisted design, robotic dispensing or layer-by-layer manufacturing are used for printing cells and biomaterials in 2D/3D spaces within a reproductive framework, differentiating this approach from historical manual processes usually used for tissue engineering. The main specificity of bioprinting is related to the difference between the printed construct and the final tissue obtained after maturation. Indeed, the living construct will change in time due to morphogenesis tissue formations mechanisms (differentiation, migration, etc . . .) which occur after printing. To get the right biological functions of the maturated tissue, the initial construct needs to strictly follow a CAD Blueprint which will ensure the right tissue self-organization pathway after printing. As a consequence, reproducibility, volume fraction and resolution have to be considered as fundamental assets for the bioprinting technology used for tissue engineering to get the right tissue, whatever its application.

Different technologies have been developed for that purpose, mainly based on nozzle solutions such as extrusion, inkjet or micro-valve, extrusion being the most prevalent one. All of them exhibit the same type of limitations as they suffer from inherent issues like clogging of print heads and from shear stress applied to cells when they flow through nozzle, resulting in a poor cell printing viability. Printing tissue aggregates with extrusion is even more difficult in regards to clogging and sedimentation due to the size of the objects to print. As a result, the needle needs to be enlarge and the resulting compactness factor is increasingly less important, typically less than 30%. Zhuang et al. (2021) Int J Bioprint. 7(4), 444 describes the limitations of spheroids printing by extrusion.

Among the other bioprinting technologies available, some are more reliable but particularly dedicated to spheroids manipulation/bioprinting: Kenzan bioprinting and Aspiration assisted bioprinting. But they also suffer from limitations preventing them to produce a highly packed tissue.

Kenzan bioprinting [Yurie et al. (2017) PLoS ONE 12(2), e0171448] is a method to assemble cellular aggregates into any desired 3D macroscopic tissue without the help of a scaffold made of collagen or hydrogel materials. Indeed, spheroids are disposed within a fine needle array where they can merge with adjacent spheroids to form a connected structure. Using appropriate alignment of needles, spheroids can be positioned in any desired 3D layout. This Kenzan technology, particularly adapted to hollow constructs, is limited in term of production flow as spheroids need to be manipulate individually with a clamp or a syringe. Furthermore, the size of the spheroids is usually large to be compatible with needle insertion. Kenzan does not allow producing dense tissue (with a high compactness factor) made of spheroids smaller than 500 μm in diameter.

Aspiration assisted bioprinting is another technique particularly adapted to spheroids manipulation and patterning [Ayan et al. (2020) Communications Physics 3, 183]. This technology enables picking and positioning aggregates in 3D through harnessing the power of aspiration forces. It operates a pipette, which is used to “pick up” spheroids from a gel or a bio-ink and “3D bioprint” them into or onto a gel substrate (receiver). This technology claims a high precision positioning and a high viability, but the aspiration needle is in contact with the spheroid, with potential contaminations and mechanical damages. Furthermore, to ensure a good aspiration, this solution is limited to manipulation of large size spheroids and is consequently limited in term of production flow. As for Kenzan, spheroids need to be handle individually with slow motion. An objective description of this technology is given in the following paper. Equally to Kenzan, aspiration based bioprinting does not allow producing dense tissue (high compactness factor) made of spheroids smaller than 500 μm in diameter.

Laser assisted bioprinting (LAB) which is a needle-free technology. It is based on the use of a pulsed laser source that generates the transfer of bio-ink micro-droplets from a target to a receiving substrate placed a few hundred micrometers to millimeters away. The main advantage of laser assisted bioprinting is its ability to deposit cells with very high resolution, up to the single cell. The absence of an orifice also ensures cell viability greater than 95%.

LAB is based on the use of a donor slide (cartridge) on which the bio-ink is spread as a thin film. Then, a scanner equipped with a f-theta lens focuses the laser beam onto the slide in order to create plasma absorption which results in bubble cavitation into the liquid and finally in jet formation from the bio-ink surface. Jets will deposit bio-ink onto the receiver substrate with a dynamic and droplet sizes depending on a large number of parameters mainly from the laser (energy, pulse duration, fluence, wavelength . . . ) and the bio-ink (thickness, viscosity, surface tension . . . ). Thus, depending on those numerous parameters the droplet volumes can vary a lot from one condition to the other. Users have to adapt their experimental conditions—laser and bio-ink parameters—to get the right droplet size and the right number of objects within the droplets. The ability of LAB to transfer large objects has already been described, particularly in solid transfer for micro-electronics. WO2018167402, describes specific conditions to print objects ranging from sub-cellular components (exosomes for example) to cells and even to large biological objects.

WO2018167400 describes a system for acquiring an image of the fluid film, for analyzing the acquired image and recognizing geometric positions of inhomogeneities and for directing the laser beam toward the position of one inhomogeneity to print it toward the receiver. WO2016097619 describes upward laser printing. WO2016097620 relates to multimodal printing.

LAB is a direct laser writing technique used to transfer a small quantity of a liquid containing living cells from a donor towards a receiver. A pulsed laser is focused on the donor composed of a transparent substrate usually coated with a thin metallic layer (10-100 nm thick) and a bio-ink (e.g. cell media, hydrogel) [Barron (2004) Biomed. Microdevices 6, 139-147]. The laser energy is largely absorbed by the metallic layer at the focused spot. When the energy is high enough, the metal is ablated and instantaneously converted into a plasma that generates a small cavitation bubble within the fluid. This bubble expands in close vicinity to the liquid-air interface until it collapses and creates a liquid jet. When the jet is sufficiently extended and close enough to the receiver, a bio-ink droplet can be deposited onto the receiver. Each printed droplet contains a defined number of cells, ranging from the single-cell level to larger numbers (several tens of cells) depending on the targeted application.

LAB is based on laser-induced forward transfer (LIFT), where the energy from a pulsed laser induces the transfer of bio-ink (e.g. cell media, hydrogel) containing cells or biomolecules from a source film onto a receiver plate in close proximity [Ringeisen et al. (2204) Tissue Engineering 10(3-4), 483-491; Ali et. al (2014) Biofabrication 6, 045001]. The droplet transfer occurs within hundreds of microseconds and with a micron-scale precision which allows for fast printing of cells with controlled patterns at a high resolution [Guillotin et al. (2010) Biomaterials 31, 7250-7256]. Movement of the receiver plate further allows the formation of specific patterns via computer-aided design. LAB has been used for printing of multiple different single cell types (e.g. rabbit carcinoma cell line, rat acinar cell line, human induced pluripotent stem cells) with maintained viability. Continued culture after printing has demonstrated that the printed cells are functional and able to differentiate and form functional tissues, e.g. pancreas model, skin and bone.

LAB technology however does not provide proper conditions to achieve dense pattern printing with high compactness factor, preserved spheroid size and shape, cell differentiation capability.

### SUMMARY OF THE INVENTION

The invention relates to methods of manufacturing a three dimensional osteochondral tissue by Laser Induced Forward Transfer (LIFT) laser printing, comprising the steps of:
a) providing a donor bio-ink comprising aggregates of cartilage and/or bone forming cells,
b) transferring cell aggregates to a receiver substrate into a patterned layer of cell aggregates by pulsed laser energy focused on the bio-ink,
wherein the laser energy is in the range of 10 to 100 μJ, the nanosecond laser operating in the NIR between 800 and 1500 nm, and
wherein cell aggregates are deposited at a surface density between 10 and 500aggregates per mm²,
c) repeating steps a) and b) to obtain further patterned layers of aggregates on previously deposited layers.

In embodiments of these methods, in step a) the ratio between the thickness of the bio-ink on the donor substrate versus the diameter of the aggregates in said bio-ink is between 3/1 and 1/1, typically 2/1.

In embodiments of these methods, the patterning is obtained by movement of the laser beam and/or by movement of the receiver substrate.

In embodiments of these methods, laser energy is adjusted to obtain droplets of between 10 or 30 nl to 400 or 500 nl, typically between 100 nl to 200 nl.

In embodiments of these methods, the viscosity of the donor ink is between 1 and 100 centipoise.

In embodiments of these methods, the laser energy is between 10 to 40 μJ for a nanosecond laser operating at 1064 nm.

In embodiments of these methods, the laser spot size has a diameter of between 25 to 50 μm.

In embodiments of these methods, the distance between donor bio-ink and receiver substrate is between 1 to 5 mm, typically between 2 to 3 mm.

In embodiments of these methods, the concentration of cell aggregates is between 10.000 and 200.000 aggregates per ml donor bio-ink.

In embodiments of these methods, a cell aggregate comprises between 100 and 500 cells, or a cell aggregate has a diameter between 50 μm and 150 μm, or between 50 and 500 μm, or between 150 and 350 μm.

In embodiments of these methods, a single aggregate is comprised in a single droplet.

In embodiments of these methods, the cartilage producing cell aggregates are spheroids comprising periosteum derived cells.

In embodiments of these methods, the spheroids have been cultivated after aggregation for a period of between 3 to 21 days, or between 5 to 9 days, typically for a period of 7 days.

In embodiments of these methods, the receiver substrate is agarose or a hydrogel. In embodiments of these methods, the donor bio-ink comprises a one or more compounds selected of the group consisting of a growth factor, a monomer of polymerizable polymer, a detectable marker such as a dye and a cell adhesion compound such as collagen or hyaluronic acid.

In embodiments of these methods, in between the repetition of steps a) and b) a compound selected from one or more the group of a growth factor, a monomer of polymerizable polymer and a cell adhesion compound is deposited on the layer of patterned aggregates.

In embodiments of these methods, the laser printing prints between 100 to 10000aggregates per second.

An aspect of the invention and its embodiments are summarized in the below statements 1 to 26:
1. A method of manufacturing a three dimensional cartilage tissue by laser assisted bioprinting, comprising the steps:
   a) providing a donor bio-ink comprising aggregates of cartilage forming cells,
   b) transferring a patterned layer of cell aggregates on a receiver substrate by pulsed laser energy focused on the bio-ink,
   c) repeating steps a) and b) to obtain further patterned layers of aggregates on previously deposited layers thereby obtaining a said three dimensional cartilage tissue, typically with a compactness factor of at least 30%, 40, 50, 60 or 70%
2. The method according to statement 1, wherein the patterning is obtained by movement of the laser beam and/or by movement of the receiver substrate.
3. The method according to statement 1 or 2, wherein laser energy is adjusted to obtain droplets of between 10 or 30 nl to 400 or 500 nl, typically between 100 nl to 200 nl.
4. The method according to any one of statements 1 to 3, wherein the viscosity of the donor ink is between 1 and 100 centipoise.
5. The method according to any one of statements 1 to 4, wherein the laser energy is between 10 to 40 μJ for a nanosecond laser operating at 1064 nm.
6. The method according any one of statements 1 to 5, wherein the laser spot size has a diameter of between 25 to 50 μm.
7. The method according to any one of statements 1 to 6, wherein the distance between donor bio-ink and receiver substrate is between 1 to 5 mm, typically between 2 to 3 mm.
8. The method according to any one of statements 1 to 7, wherein the concentration of cell aggregates is between 10.000 and 200.000 aggregates per ml donor bio-ink.
9. The method according to any one of statements 1 to 8, wherein a cell aggregate comprises between 100 and 500 cells.
10. The method according to statement any one of statements 1 to 9, wherein a cell aggregate has a diameter between 50 μm and 150 μm, or between 50 and 500 μm, or between 150 and 350 μm.
11. The method according to any one of statements 1 to 10, wherein the cartilage producing cell aggregates are spheroids.
12. The method according to statement 11, wherein the spheroids comprise periosteum derived cells.
13. The method according to statement 11 or 12, wherein the spheroids have been cultivated after aggregation for a period of between 3 to 21 days, or between 5 to 9 days, typically for a period of 7 days.
14. The method according to any one of statements 1 to 13, wherein the receiver substrate is agarose or a hydrogel.
15. The method according to any one of statements 1 to 14, wherein the donor bio-ink comprises a viscosity enhancing compound.
16. The method according to any one of statements 1 to 15, wherein the donor bio-ink comprises a compound selected from one or more the group of a growth factor, a monomer of polymerizable polymer a detectable marker such as a dye and a cell adhesion compound such as collagen or hyaluronic acid.
17. The method according to any one of statements 1 to 15, wherein in between the repetition of steps a) and b) a compound selected from one or more the group of a growth factor, a monomer of polymerizable polymer and a cell adhesion compound is deposited on the layer of patterned aggregates.
18. The method according to any one of statements 1 to 17, wherein laser printing can is coupled with other printing modalities to transfer biomaterials, such as collagen or Hyaluronic acid, into said cartilage tissue.
19. The method according to any one of statements 1 to 18, wherein the compactness factor is greater than 50% to produce articular cartilage.
20. The method according to any one of statements 1 to 19, wherein the cartilage tissue architecture is be divided into several zones with specific cell types aggregates and compactness factor.
21. The method according to any one of statements 1 to 20, wherein the laser printing prints between 100 to 10000 aggregates per second. This gives the possibility to print a large number of aggregates per second in high throughput manner, reducing the time to produce said cartilage tissue in order to keep it alive all along the fabrication process.
22. The method according to any one of statements 1 to 21, wherein the contact-less transfer by laser printing prevents cell degradation and is compatible with GMP requirements for clinical applications.
23. The method according to any one of statements 1 to 22, wherein post printing viability of cells is greater than 80% to get a functional cartilage tissue.
24. The method according to any one of statements 1 to 23, wherein the cell aggregates remain fully functional after laser printing with chondrogenic differentiation capability preservation.
25. The method according to any one of statements 1 to 24, wherein the donor substrate is a fluidic chip able automatizing bio-ink refill.
26. The method according to any one of statements 1 to 25, wherein the ratio between the bio-ink thickness deposited onto the donor substrate and the diameter of the aggregate in the range of 1 to 3. This allows aggregate transfer by fluid transport and a low laser energy level to avoid cell damage.

An aspect of the invention and its embodiments are summarized in the below statements 27 to 53:
27. A method of manufacturing a three dimensional cartilage tissue by bioprinting, comprising the steps:
   a) providing a donor bio-ink comprising aggregates of cells,
   b) transferring a patterned layer of cell aggregates on a receiver substrate,
   c) repeating steps a) and b) to obtain further patterned layers of aggregates on previously deposited layers thereby obtaining a said three dimensional cartilage tissue, typically with a compactness factor of at least 30%, 40, 50, 60 or 70%.
28. The method according to statement 27, wherein bioprinting is based on a laser assisted bioprinting technology.
29. The method according to statement 28, wherein the patterning is obtained by movement of the laser beam and/or by movement of the receiver substrate.
30. The method according to statement 28 or 29, wherein laser energy is adjusted to obtain droplets of between 10 or 30 nl to 400 or 500 nl, typically between 100 nl to 200 nl.
31. The method according to any one of statement 28 to 30, wherein the laser energy is between 10 to 40 μJ for a nanosecond laser operating at 1064 nm.
32. The method according to any one of statements 28 to 31, wherein the laser spot size has a diameter of between 25 to 50 μm.
33. The method according to any one of statements 27 to 32, wherein the distance between donor bio-ink and receiver substrate is between 1 to 5 mm, typically between 2 to 3 mm.
34. The method according to any one of statements 27 to 33, wherein the viscosity of the donor ink is between 1 and 100 centipoise.
35. The method according to any one of statements 27 to 34, wherein the concentration of cell aggregates is between 10.000 and 200.000 aggregates per ml donor bio-ink.
36. The method according to any one of statements 27 to 35, wherein a cell aggregate comprises between 100 and 500 cells.
37. The method according to any one of statements 27 to 36, wherein a cell aggregate has a diameter between 50 μm and 150 μm, or between 50 and 500 μm, or between 150 and 350 μm.
38. The method according to any one of statements 27 to 37, wherein the cell aggregates are spheroids.
39. The method according to any one of statements 27 to statement 38, wherein the spheroids comprise periosteum derived cells.
40. The method according to statement 38 or 39, wherein the spheroids have been cultivated after aggregation for a period of between 3 to 21 days, or between 5 to 9 days, typically for a period of 7 days.
41. The method according to any one of statements 27 to 40, wherein the receiver substrate is agarose or a hydrogel.
42. The method according to any one of statements 27 to 41, wherein the donor bio-ink comprises a viscosity enhancing compound.
43. The method according to any one of statements 27 to 42, wherein the donor bio-ink comprises a compound selected from one or more the group of a growth factor, a monomer of polymerizable polymer a detectable marker such as a dye and a cell adhesion compound such as collagen or hyaluronic acid.
44. The method according to any one of statements 27 to 42, wherein in between the repetition of steps a) and b) a compound selected from one or more the group of a growth factor, a monomer of polymerizable polymer and a cell adhesion compound is deposited on the layer of patterned aggregates.
45. The method according to any one of statement 28 to 44, wherein laser printing is be coupled with other printing modalities to transfer biomaterials such as collagen or Hyaluronic acid, into said cartilage.
46. The method according to any one of statements 27 to 45, wherein the compactness factor is greater than 50% to produce articular cartilage.
47. The method according to any one of statements 27 to 46, wherein the cartilage tissue architecture is divided into several zones with specific cell types aggregates and compactness factor.
48. The method according to any one of statements 28 to 47, wherein laser printing gives the possibility to print a large number of aggregates per second in high throughput manner, from several hundreds to several thousands, reducing the time to produce said cartilage tissue in order to keep it alive all along the fabrication process.
49. The method according to any one of statements 28 to 48, wherein the contact-less transfer by laser printing prevents cell degradation and is compatible with GMP requirements for clinical applications.
50. The method according to any one of statements 27 to 49, wherein post printing viability of cells is greater than 80% to get a functional cartilage tissue.
51. The method according to any one of statements 28 to 50, wherein the cell aggregates remain fully functional after laser printing with chondrogenic differentiation capability preservation.
52. The method according any one of statements 27 to 51, wherein the donor substrate is based on a fluidic chip able to automatize bio-ink refill.
53. The method according to any one of statements 27 to 52, wherein the ratio between the bio-ink thickness deposited onto the donor substrate and the diameter of the aggregate in the range of 1 to 3. This allows aggregate transfer by fluid transport and a low laser energy level to avoid cell damage.

In the methods of the invention the laser energy can be adjusted to obtain droplets of a desired size, typically between 10 or 30 nl to 400 or 500 nl, or between 100 nl to 200 nl.

In the methods of the present invention the viscosity of the donor bio-ink is between 1 and 100 centipoise, and can be adjusted using a viscosity enhancing compound. The donor bio-ink further can comprises a compound selected from one or more the group of a growth factor, a monomer of polymerizable polymer a detectable marker such as a dye and a cell adhesion compound such as collagen or hyaluronic acid. These compounds will then be deposited on the donor substrate together with the cell aggregates.

Alternatively or in addition a compound selected from one or more the group of a growth factor, a monomer of polymerizable polymer and a cell adhesion compound is applied on a layer of printed aggregates.

Other printing modalities next to LIFT can be used to transfer biomaterials, such as collagen or hyaluronic acid, onto or around the printed aggregates.

In the methods of the present invention is between 10 to 100 μL or 10 to 75 μJ or 10 to 40 μJ for a nanosecond laser operating in the near infrared range between 800and 1500 or between 900 and 1250 nm or between 1050 and 1100 nm, more typically at 1064 nm.

In the methods of the present invention the diameter of the laser spot size can be adjusted to diameter of between 10 to 75 μm or between 25 to 50 μm.

In the methods of the invention, the distance between donor bio-ink and receiver substrate is between 1 to 5 mm, typically between 2 to 3 mm.

In the methods of the invention, the concentration of cell aggregates in the bio-ink is between 10.000 and 200.000 aggregates per ml donor bio-ink, or 10.000 and 100.000 aggregates per ml donor bio-ink, or 10.000 and 50.000 aggregates per ml donor bio-ink.

In the methods of the invention, a cell aggregate typically comprises between 100 and 500 cells, or between 200 and 300 cells.

In the methods of the invention, a cell aggregate typically has a diameter between 50 μm and 150 μm, or between 50 and 500 μm, or between 150 and 350 μm.

Examples of cartilage producing cell aggregates used in these methods are spheroids, for example, spheroids comprising periosteum derived cells.

Such spheroids have been typically cultivated after aggregation and prior to bioprinting for a period of between 3 to 21 days, or between 5 to 9 days, typically for a period of 7 days.

In the methods of the present invention the receiver substrate can be for example is agarose or a hydrogel.

In the methods of the present invention laser printing can print between 100 to 10000aggregates per second. This gives the possibility to print a large number of aggregates per second in high throughput manner, reducing the time to produce said cartilage tissue in order to keep it alive all along the fabrication process.

The methods of the present invention allow a contact-less transfer by laser printing prevents cell degradation and is compatible with GMP requirements for clinical applications.

The methods of the present invention allow obtaining a post printing viability of cells is greater than 80% to get a functional cartilage tissue.

With the methods of the present invention cartilage forming cell aggregates remain fully functional after laser printing with chondrogenic differentiation capability preservation.

In methods of the present invention the bio-ink thickness deposited onto the donor substrate and the diameter of the aggregate in generally in the range of 1 to 3. This allows aggregate transfer by fluid transport and a low laser energy level to avoid cell damage.

In the methods of the invention, patterning can obtained by movement of the laser beam and/or by movement of the receiver substrate.

### DETAILED DESCRIPTION

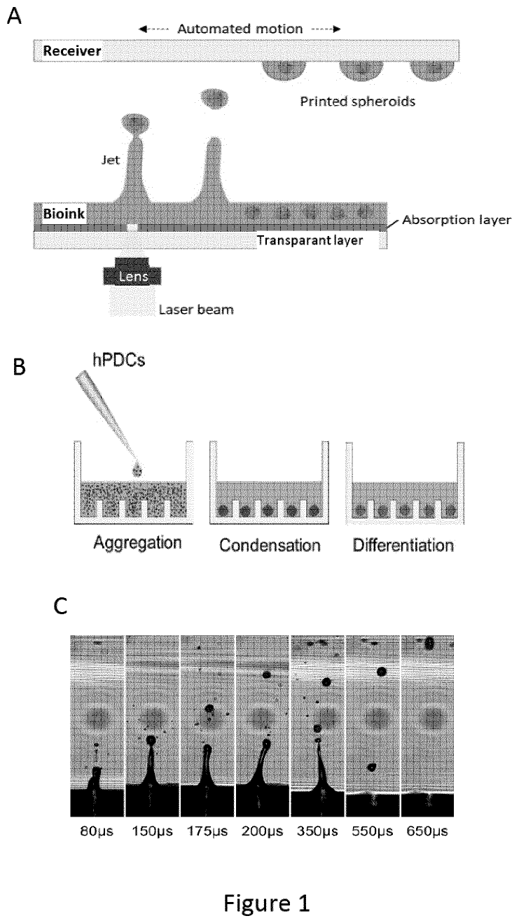

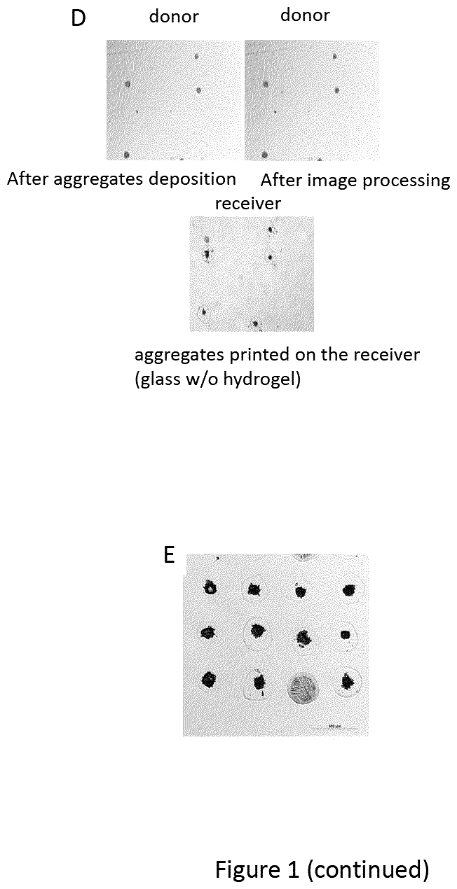

FIG. 1. (a) Schematic overview of the laser-assisted bioprinting (LAB) setup. A focused laser beam hits the absorption layer which generates a bubble cavitation into the bio-ink (in this work CM). The cavitation bubble expands and a jet is formed with a droplet containing the spheroid is transferred onto the receiver plate. (b) Schematic overview and brightfield (BF) images of spheroid formation. (c) Time-resolved imaging of laser-induced jet formation in LAB of spheroids. The droplet transferring the spheroid is released at 150-175 μm. Images were taken with bio-ink at 4 mm distance to the receiver. (d) Image-based selection of spheroids to print. (e) Spheroids printed in a pre-defined grid.

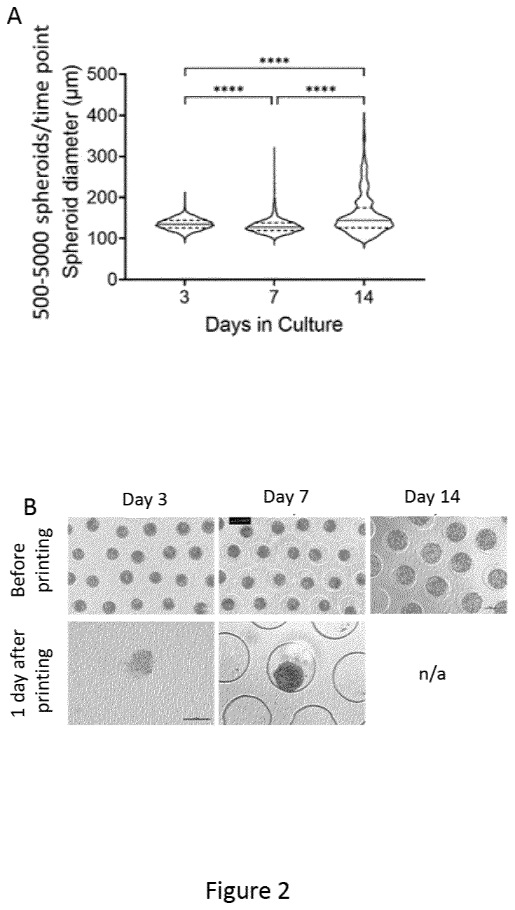

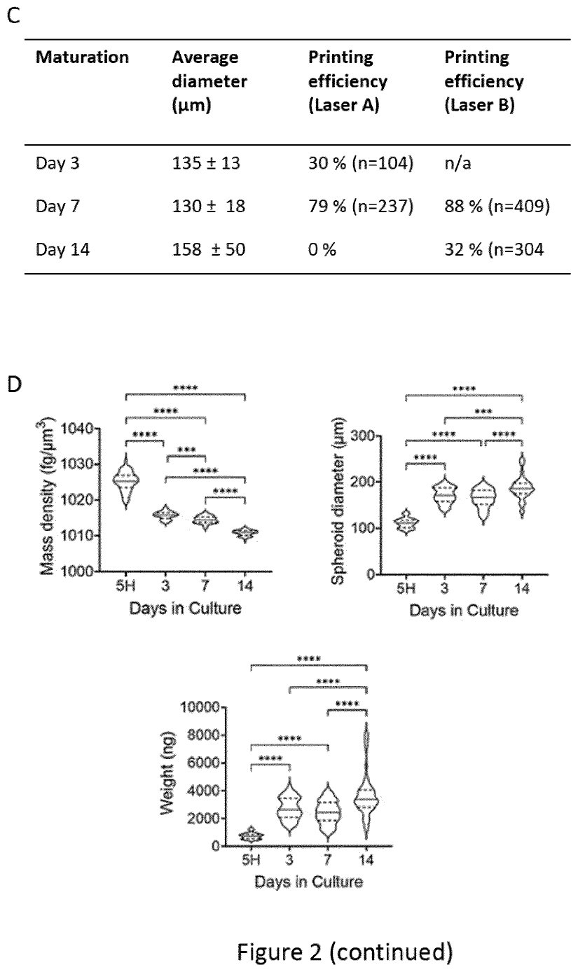

FIG. 2. (a) Spheroid diameter from the different time points. Graph shows violin plot with median (red) and quartiles (dashed). (b) Brightfield images of spheroids before and 1 day after LAB. (c) Table shows spheroid diameter and printing efficiency for the different spheroid maturations and lasers. (d) Mass density, spheroid diameter and weight of spheroids of different maturation (5h, 3d, 7d and 14d) measured with W8 microfluidic device. Graph shows violin plot with median and quartiles.

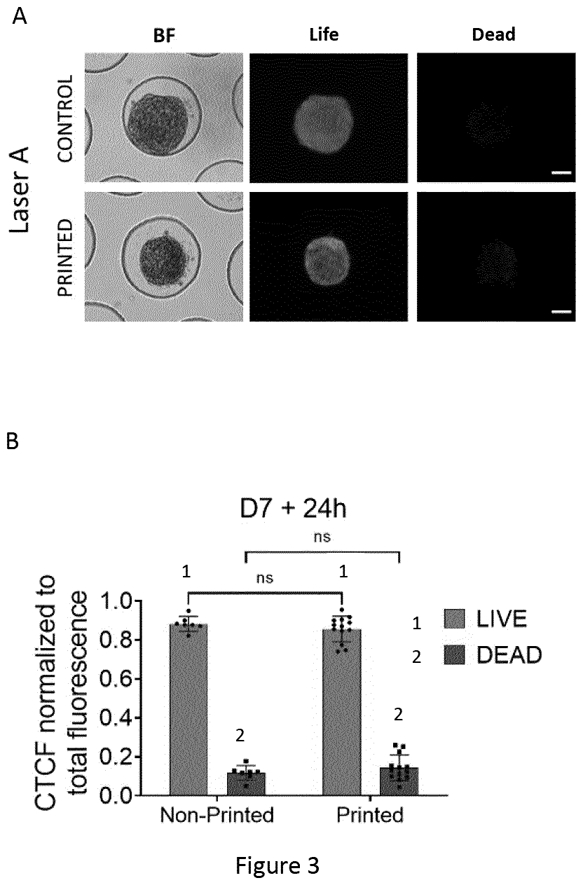

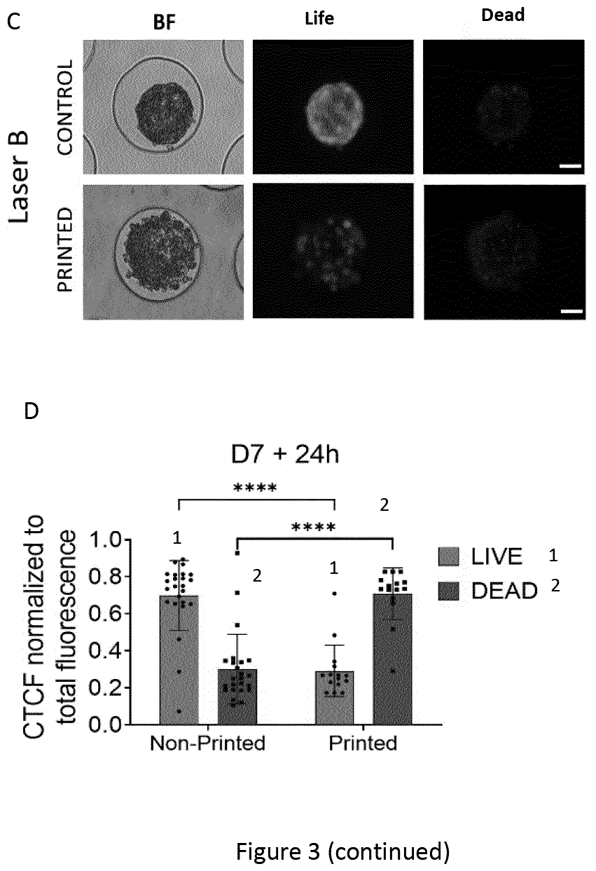

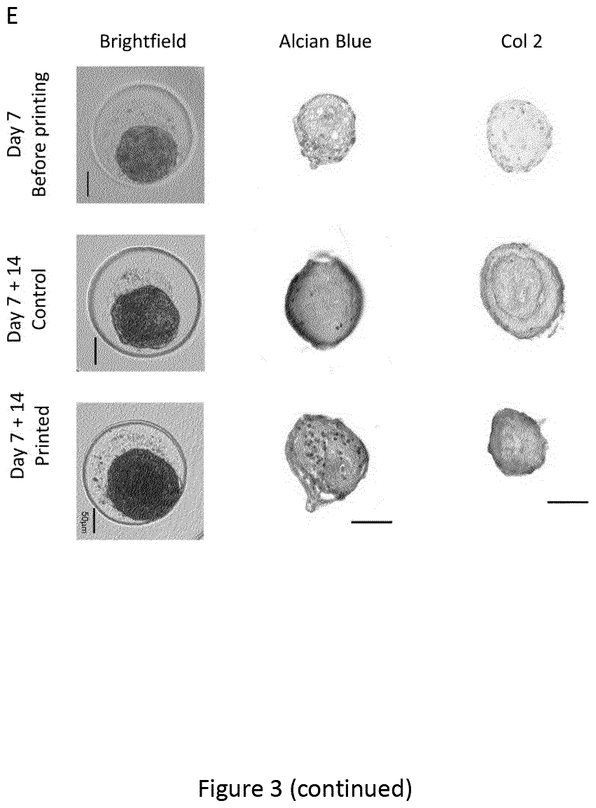

FIG. 3. (a) Viability staining of day 7 spheroids printed with Laser A and (b) its semi-quantification (7-13 spheroids quantified; graph show mean ±SD and each point represents one spheroid). (c) Viability staining of day 7 spheroids printed with Laser B and (d) its semi-quantification (7-13 spheroids quantified; graph show mean ±SD and each point represents one spheroid). (e) Brightfield images, alcian blue histological staining and Col 2 immunostaining of spheroids before printing and 14 days after printing. Scale bars represent 50 μm.

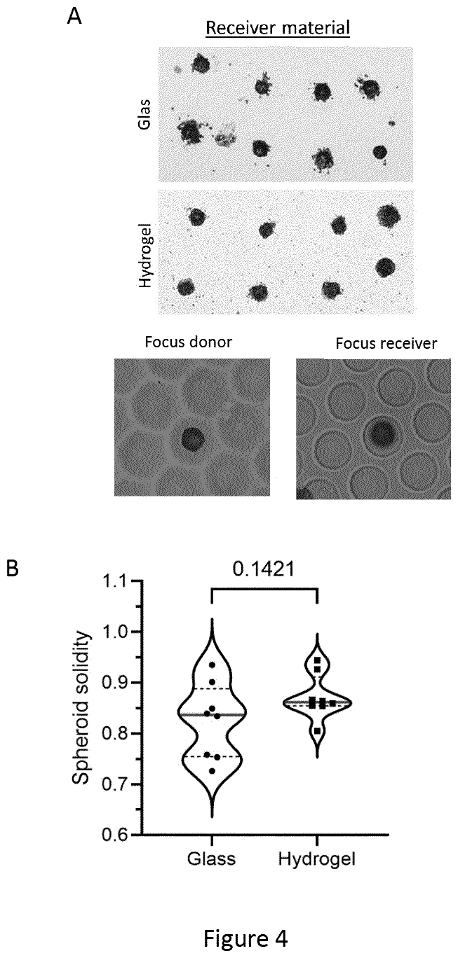

FIG. 4. (a) Photos of spheroids printed onto different receiver material: glass and hydrogel. (b) Quantification of spheroid solidity after printing onto the different receiver materials. Graph shows violin plot with median and quartiles; each point represents one spheroid.

“Bio-inks” are materials used to produce engineered/artificial live tissue using 3D printing. These inks are mostly composed of the cells that are being used, but are often used in tandem with additional materials that envelope the cells. The combination of cells and usually biopolymer gels are typically refer to as a bio-ink. Apart from the cells the bio-ink may comprise:
polysaccharides such as alginate, gellan gum, or agarose,
protein-based material such as gelatin or collagen,
synthetic Polymers, such as Pluronics, PEG,
decellularized ECM.

In the context of the present invention the bio-ink can also contain one or more of a growth factor, a monomer of polymerizable polymer, a detectable marker such as a dye or a cell adhesion compound such as collagen or hyaluronic acid.

“Aggregates” in the context of the present invention relates to cells in a scaffold free environment, which are attached to each other. “Spheroids” in the context of the present invention relates to cell aggregates wherein the attachment of the cells is enhanced by the presence of extracellular matrix. Aggregates and spheroids may exist of one single cell type or of different cell types. A particular embodiment of spheroids comprises periosteum derived cells or iPS derived cell with bone or cartilage forming properties.

“Compactness Factor (CF)” in the context of the present invention refers to the density of the packing of aggregates in a 3D printed tissue. For example, a CF of 30% refers to a 3D printed tissue wherein 30 % of the volume of the tissue is occupied by the cell aggregates. The remaining 70 % comprises remnants of the bio-ink and optional other agents delivered during the bioprinting process.

“LAB” is a direct laser writing technique used to transfer a small quantity of a liquid containing living cells from a donor towards a receiver. A pulsed laser is focused on the donor composed of a transparent substrate usually coated with a thin metallic layer (10-100 nm thick) and a bio-ink (e.g. cell media, hydrogel) [Barron (2004) Biomed. Microdevices 6, 139-147]. The laser energy is largely absorbed by the metallic layer at the focused spot. When the energy is high enough, the metal is ablated and instantaneously converted into a plasma that generates a small cavitation bubble within the fluid. This bubble expands in close vicinity to the liquid-air interface until it collapses and creates a liquid jet. When the jet is sufficiently extended and close enough to the receiver, a bio-ink droplet can be deposited onto the receiver. Each printed droplet contains a defined number of cells, ranging from the single-cell level to larger numbers (several tens of cells) depending on the targeted application.

LAB (Laser assisted bioprinting) is based on laser-induced forward transfer (LIFT), where the energy from a pulsed laser induces the transfer of bio-ink (e.g. cell media, hydrogel) containing cells or biomolecules from a source film onto a receiver plate in close proximity [Ringeisen et al. (2204) Tissue Engineering 10(3-4), 483-491; Ali et. al (2014) Biofabrication 6, 045001]. The droplet transfer occurs within hundreds of microseconds and with a micron-scale precision which allows for fast printing of cells with controlled patterns at a high resolution [Guillotin et al. (2010) Biomaterials 31, 7250-7256]. Movement of the receiver plate further allows the formation of specific patterns via computer-aided design.

The present invention relates to the generation of cartilage tissue by laser assisted bioprinting of cell aggregates with the following value attributes.

The method of the present invention results in a tissue that has an number advantageous properties:
a high post printing viability of more than 80 or 90 % a preserved spheroid size and shape without cell losses or spheroid breakage by selecting parameters such as ink formulation, ink thickness, laser energy, spheroid diameter and focal spot size.
cell differentiation capability preserved after printing
a precise and predetermined deposition of aggregates. This allows a dense patterning leads to a high compactness factor (>30% for the transient and >50% for the articular).
a precise and predetermined deposition of cell aggregates

In the case of spheroid printing by LAB technology, bio-ink deposited onto the donor slide is typically first imaged in order to localize the spheroids in 2D (via a imaging system coupled to processing algorithms). In a second step, a scanner will target the corresponding zones where spheroids are located to shoot them with the laser. Herein LAB has the ability to transfer nearly 100% of spheroids from the bio-ink towards specific locations of the receiver substrate.

Alternatively, it is possible to shoot directly spheroids dispersed in the bio-ink film without imaging step when spheroid concentrations are large enough to cover a large part of the donor surface. Under these conditions, the printing process is significantly faster but may suffer from a slightly lower transfer efficiency related to the probability of targeting a spheroid in a dispersed solution.

The thickness of the bio-ink film is optimized to reduce laser energy needed for jet generation to prevent thermal effects or cell damage. The thickness of the bio-ink for prior art single cell suspension printing is typically in the range of 100 to 200 μm. In the case of aggregate printing the ratio between the bio-ink thickness deposited onto the donor substrate and the diameter of an aggregate is typically in the range of 1 to 3 to allow aggregate transfer by fluid transport and to provide a laser energy level to avoid cell damage.

To obtain a dense packing of the printed spheroid, the amount of bio-ink that is transferred with the spheroids is kept to a minimum. This volume depends on parameters such as laser energy, ink rheological properties, distance between the donor and the receiver and size of the spheroid. To obtain such dense packing, the volume of bio-ink in the donor solution is typically between 1, 2, 5, 10 up to 20, 30, 40, 50 or 60 % of the volume of cell spheroids. All ranges defined by the above lower and upper values are herewith explicitly disclosed.

The present invention discloses the printing of CBBs such as cartilaginous spheroids. Human periosteum derived cells (hPDCs) form spheroids with an increasing amount of cartilaginous extracellular matrix (ECM) when cultured in non-adherent microwells and chondrogenic media [Nilsson Hall et al. (2020) Adv. Sci. 7, 1-16]. LAB was performed on hPDC spheroids of different maturity, going from day 3 (mainly cells), day 7 (cells and low ECM) and day 14 (cells and ECM). Printing parameters were defined using fixated spheroids followed by viable spheroids. Finally, viability and histological staining was used to assess functionality after printing.

### Example 1. Materials and Methods

Cell culture

Human periosteum derived cells (hPDCs) were isolated through digestion of five donors (14±3 years old) as described in Eyckmans et al. (2010) J. Cell. Mol. Med. 14, 1845-1856. Briefly, the periosteal biopsies were washed and digested in type IV collagenase (440 units/mg, Invitrogen, BE) dissolved in growth medium (high-glucose Dulbecco's modified Eagle's medium with sodium pyruvate (DMEM, Invitrogen, BE) supplemented with 10% fetal bovine serum (FBS, Hyclone), and an antibiotic-antimycotic solution (100 μg/ml streptomycin, 100 units/ml penicillin and 0.25 μg/ml amphotericin B, Invitrogen, BE)). Next, the digested cells were pooled together to create a cell pool. The cell pool was expanded until passage 9 in growth medium at 37° C., 5% CO₂ and 95% humidity. Growth medium was changed three times per week until 90% confluency when the cells were harvested with TrypLE™ Express (Life Technologies, UK).

### Generation of Microwells and Spheroids

Agarose microwells were obtained by a double-molding procedure using soft lithography techniques as described in Nilsson Hall et al. (2021) Biofabrication 13(4). Briefly, a SU-8 wafer was fabricated to produce a polydimethylsiloxane (PDMS) mold containing pillars with a diameter of 200 μm. Next, the monomer and curing agent (Dow Corning, Midland, MI, USA) were mixed, degassed and casted over the SU-8wafer to create PDMS pillars. The PDMS pillars were removed from the wafer after 2 h bake at 65° C. To create the agarose microwells, 3% UltraPure™ agarose (Thermo Fisher) was poured over the PDMS mold and let to cool down. The agarose microwells were punched out (1.8cm² ) and placed in a 24-well plate and sterilized under UV before use for spheroid formation. hPDCs harvested as described above were resuspended in a serum-free chondrogenic media (CM) composed of LG-DMEM (Gibco) supplemented with 1% antibiotic-antimycotic (100 units/mL penicillin, 100mg/mL streptomycin and 0.25 mg/mL amphotericin B), 100 nM dexamethasone, 1 mM ascorbate-2 phosphate, 40 g/mL proline, ITS+Premix Universal Culture Supplement (Corning) (including 6.25 μg/mL insulin, 6.25 μg/mL transferrin, 6.25 μg/mL selenious acid, 1.25 μg/mL bovine serum albumin (BSA), and 5.35 μg/mL linoleic acid), 20 μM of Rho-kinase inhibitor Y27632 (Axon Medchem), 100 ng/ml GDF5 (PeproTech), 100 ng/ml BMP-2 (INDUCTOS®), 10 ng/ml TGFβ1 (PeproTech), 1 ng/ml BMP-6 (PeproTech) and 0.2 ng/ml FGF-2 (R&D systems) [Nilsson Hall et al. (2020) Adv. Sci. 7, 1-16; Mendes et al. (2016) Tissue Eng. Part C Methods 22, 473-86]. 500.000 cells were seeded in each well containing approximately 2000microwells resulting in the generation of spheroids with approximately 250 cells/spheroids with 250 000cells/mL CM. The seeded spheroids were cultured at 37° C., 5% CO₂ and 95% humidity with half amount media change every 3-4 days.

### Laser-Assisted Bioprinting of Spheroids and Post Culture

The LAB workstation used has previously been described by Guillemot et al. (2010) Acta Biomater, 6 2494-2500.
Bioprinter: LAB workstation setup

### Laser Characteristics

Laser A: Satsuma 5W, 25 μJ (Amplitude Systems, France), 1030 nm, ultra short pulses from 350 fs to 10 ps with several μJ to several tens of μJ of energy/pulse
Laser B: YLPN-0.7-2×200-20-SM, Ytterbium pulsed fiber laser (IPG, USA), 1064nm, longer pulses, from 2 to 200ns, with several μJ to several tens of μJ of energy/pulse
Type of lens: F-theta lens with 100 mm focal distance fit for laser scanning.
Typical spot size at focal plane is in the range of 30 to 35um in diameter
Donor preparation, gold film (absorption layer): 700 μm thick glass slide, transparent at laser wavelength, coated with 20 nm gold layer
Used cell culture media (CM) as bio-ink
Agarose microwells (3% agarose) as receiver
DDR 2-3 mm: distance between donor and receiver achieved thanks to robotic arm
   Measurement of Spheroid Density Spheroids at defined time points (5h, 3d, 4d and 14d) were washed in Dulbecco's phosphate-buffered saline (DPBS) (Lonza, USA) and fixated in 4% paraformaldehyde (PFA) (Merck, Germany) for 1h at room temperature followed by washing in PBS. Next, samples were resuspended to a concentration lower than 100 spheroids/mL and analyzed by the company (CELLDYNAMICS I.S.R.L., Italy) as described in Cristaldi et al. (2020) Micromachines 11, 1-13. 29-50 spheroids per time point were analyzed.

### Viability Staining and Semi-Quantification

Cell viability in spheroids was assessed with LIVE/DEAD® Viability/Cytotoxicity Kit (Invitrogen, USA) for mammalian cells by following the manufacturer's protocol. Briefly, spheroids were washed with PBS, where after they were incubated in 2 μM Calcein AM and 4 μM Ethidium homodimer-1 for 30 min at 37° C., 5% CO₂ and 95% humidity. Stained spheroids were visualized with Nikon Ti-S with fluorescence measurement capability, equipped with a color camera (×10 objective). Next, the viability was semi-quantified using ImageJ by quantification of corrected total fluorescence (CTCF) normalized to total fluorescence [Schneider et al. (2012) Nat. Methods 9, 671-675; Ansari et al. (2013) Methods Cell Biol. 113, 295-309.] Briefly, fluorescence intensity was quantified and normalized to the background [integrated density of spheroid—(spheroid area x mean fluorescence of background)] followed by normalization to the total fluorescence (live+dead).

### Histological Staining

Spheroids in agarose molds were fixated in 4% PFA followed by embedding in paraffin. Samples were sectioned at 5 μm for subsequent alcian blue (pH 1, Sigma-Aldrich, USA) with nuclear fast red (Lab vision, USA) counterstain and safranin O staining (Klinipath, Netherlands) with fast green (Klinipath, Netherlands) counterstain according to Fernando et al. (2017) J. Tissue Eng. Regen. Med. 1-14. Collagen type II immunostaining (Col 2) was performed via antigen retrieval (1 mg/mL pepsin in 0.02 M HCl), washes (0.1% Tween20), quenching in 3% H202 and blocking followed by primary anti-Col2 antibody (dilution 1:50, AB761, Merck Millipore) incubation overnight at 4° C. Next, slides were blocked (5% bovine serum albumin) and incubated with secondary anti-rabbit antibody (dilution 1:500, 111-035-003, Jackson ImmunoResearch, UK) followed by visualization with DAB (K3468, Dako, US) and counterstaining with haematoxylin (Sigma-Aldrich, USA). Stained sections were imaged with a Leica M165 FC microscope (Microsystems, BE).

### Histological Staining

Statistical analysis was performed using GraphPad Prism 9 software (GraphPad Software, Inc., La Jolla, CA) and represented as mean±S.D. if nothing else was noted. Analysis of Variance followed by Tukey post hoc test was used to find the significant differences between the means of the different groups with p<0.05: * p<0.01: ** and p<0.001: ***.

### Example 2. Laser-Assisted Bioprinting Using Fixated Spheroids

Laser-assisted bioprinting transfer bio-ink containing cells from an absorptive layer onto a receiver plate with a focused laser (FIG. 1a). Fixated spheroids were used to define suitable bio-printing settings. Spheroids were generated in non-adherent agarose microwells by drop-seeding progenitor cells which sedimented and aggregated to form spheroids (FIG. 1b). Spheroids containing mainly cells were differentiated in CM resulting in deposition of cartilaginous extracellular matrix (ECM) and formation of microtissues, as described in Nilsson Hall et al. (2020) Adv. Sci. 7, 1-16. Fixated day 7 microtissues containing both cells and ECM were used to define bioprinting parameters. The main parameters that were controlled consisted of the laser energy, laser focus spot (ablated gold surface) and deposited jet volume to achieve the formation of a jet large enough to print a spheroid (100-150 μm). Spheroid printing was possible by increasing the maximum jet volume by a factor 15 and droplet diameter by 10, as compared to printing of single cells (10 μm) [Zhang et al. (2021) Funct. Mater. 31 2102777]. This was achieved by raising the energy to 23 μJ, deposited volume to 30 μL and ablated gold surface to 7000 μm² (FIG. 1c). First, a very thin, high speed jet appeared (−80 μs) followed by a thicker jet at lower speed (80-150 μs). The thicker jet destabilized and created a droplet containing the spheroid (175 μs) which reached the receiver surface resulting in droplet printing of the spheroid. These settings resulted in the formation of a jet with a diameter of about 150 μm to 200 μm and spheroid transfer success of 94% (37 spheroids). A dedicated software was used to determine spheroids to be transferred allowed computer-aided design (CAD) printing with high precision (FIG. 1d). The possibility to direct the laser towards specific spheroids allowed printing with precision to create patterns with fixated spheroids (FIG. 1e).

### Example 3. Laser-Assisted Bioprinting of Viable Spheroids

The defined printing parameters were then tested in viable spheroids of different maturity. Day 3 spheroids contained mainly cells and limited amount of ECM while day 7 and day 14 contained cells and ECM with more advanced chondrogenic maturity. Differences in spheroid size were also detected based on diameter, with day 7 spheroids being the smallest (FIG. 2a). Day 3 spheroids were printed with a success rate of 30% but did not retain their shape 1 day after printing (FIG. 2b-c, Laser A). Hence, the changed parameters, as compared to printing single cells, resulted in a larger jet which allowed the printing of day 3 spheroids but the impact was too strong on the immature day 3 spheroids which mainly contained cells. In contrary, the day 7 spheroids which also contained ECM secreted by the cells themselves, were successfully printed with a success rate of 79% and with retained shape one day after printing (FIG. 2b-c, Laser A). Finally, the day 14 spheroids were test printed using the laser A without success (FIG. 2b-c, Laser A). Consequently, a laser with longer pulses (Laser B) was tested to enable printing of the more mature day 14 spheroids. This laser demonstrated promising spheroid printing and allowed printing of day 14 spheroids with 32% success rate and day 7 spheroids with 88% success rate (FIG. 2c). However, the relatively low success rate of day 14 spheroid printing was still evident. A difference in size was present between the spheroids of different time points and it was hypothesized that the size and weight could be an explanation for the difference in printing efficiency. Therefore, the spheroid mass density, diameter and weight were measured using a microfluidic device [Cristaldi et al. (2020) Micromachines 11, 1-13]. Interestingly, 5-hour spheroids had the highest mass density followed by a continuous decrease in mass density with increased differentiation time (FIG. 2d). This difference may be attributed to the difference in spheroid composition with regard to cells and ECM since 5-hour spheroids are mainly composed of aggregated cells while the day 14spheroids contain more ECM but less cells (based on histology in Nilsson Hall et al. (2020) Adv. Sci. 7, 1-16). Although the day 14 spheroids had a lower mass density, they were significantly larger than earlier spheroids and their total weight were subsequently also significantly higher (FIG. 2d).

### Example 4. Spheroid Functionality After Printing

Based on the printing efficiency, day 7 spheroids were chosen for further experiments. A viability staining was performed 24 hours after laser-assisted printing. Semi-quantification demonstrated that printing using laser A resulted in similar viability as compared to non-printed spheroids (FIG. 3a-b). However, laser B resulted in a higher degree of cell death after printing (FIG. 3c-d). Hence, printing day 7 spheroids using laser A was chosen as suitable parameters for printing spheroids with the current set-up.

To ensure spheroid functionality after printing, printed spheroids were differentiated towards the chondrogenic lineage an additional 14 days and stained for detection of cartilaginous ECM. An increase in alcian blue, specific for glycosaminoglycans (GAG), was observed in day 7+14 spheroids for both control (non-printed) and printed spheroids (FIG. 3e). This increase in collagenous ECM was also detected with immunostaining specific for collagen type 2 (Col 2). These data demonstrate that the spheroids were viable after laser-assisted bioprinting and were able to undergo continued chondrogenic differentiation.

1. A method of manufacturing a three dimensional osteochondral tissue by Laser Induced Forward Transfer (LIFT) laser printing, comprising the steps of:
a) providing a donor bio-ink comprising aggregates of cartilage and/or bone forming cells,
b) transferring cell aggregates to a receiver substrate into a patterned layer of cell aggregates by pulsed laser energy focused on the bio ink, wherein the laser energy is in the range of 10 to 100 μJ, the nanosecond laser operating in the NIR between 800 and 1500 nm, and wherein cell aggregates are deposited at a surface density between 10 and 500 aggregates per mm²,
c) repeating steps a) and b) to obtain further patterned layers of aggregates on previously deposited layers.

2. The method according to claim 1, wherein in step a) the ratio between the thickness of the bio-ink on the donor substrate versus the diameter of the aggregates in said bio-ink is between 3/1 and 1/1, typically 2/1.

3. The method according to claim 1 or 2, wherein the patterning is obtained by movement of the laser beam and/or by movement of the receiver substrate.

4. The method according to any one of claims 1 to 3, wherein laser energy is adjusted to obtain droplets of between 10 or 30 nl to 400 or 500 nl, typically between 100 nl to 200 nl.

5. The method according to any one of claims 1 to 4, wherein the viscosity of the donor ink is between 1 and 100 centipoise.

6. The method according to any one of claims 1 to 5, wherein the laser energy is between 10 to 40 μJ for a nanosecond laser operating at 1064 nm.

7. The method according any one of claims 1 to 6, wherein the laser spot size has a diameter of between 25 to 50 μm.

8. The method according to any one of claims 1 to 7, wherein the distance between donor bio ink and receiver substrate is between 1 to 5 mm, typically between 2 to 3 mm.

9. The method according to any one of claims 1 to 8, wherein the concentration of cell aggregates is between 10.000 and 200.000 aggregates per ml donor bio ink.

10. The method according to any one of claims 1 to 9, wherein a cell aggregate comprises between 100 and 500 cells, or wherein a cell aggregate has a diameter between 50 μm and 150 μm, or between 50 and 500 μm, or between 150 and 350 μm.

11. The method according to any one of claims 1 to 10, wherein a single aggregate is comprised in a single droplet.

12. The method according to any one of claims 1 to 11, wherein the cartilage producing cell aggregates are spheroids comprising periosteum derived cells.

13. The method according to claim 12, wherein the spheroids have been cultivated after aggregation for a period of between 3 to 21 days, or between 5 to 9 days, typically for a period of 7 days.

14. The method according to any one of claims 1 to 13, wherein the receiver substrate is agarose or a hydrogel.

15. The method according to any one of claims 1 to 14, wherein the donor bio ink comprises a one or more compounds selected of the group consisting of a growth factor, a monomer of polymerizable polymer, a detectable marker such as a dye and a cell adhesion compound such as collagen or hyaluronic acid.

16. The method according to any one of claims 1 to 15, wherein in between the repetition of steps a) and b) a compound selected from one or more the group of a growth factor, a monomer of polymerizable polymer and a cell adhesion compound is deposited on the layer of patterned aggregates.

17. The method according to any one of claims 1 to 16, wherein the laser printing prints between 100 to 10000 aggregates per second.
